(19) United States
(12) Patent Application Publication
(10) Pub. No.: US 2003/0173527 A1
(43) Pub. Date: Sep. 18, 2003

(54) Focused ion beam system and machining method using it

(76) Inventors: Tatsuya Adachi, Chiba (JP); Toshiaki Fujii, Chiba (JP); Yasuhiko Sugiyama, Chiba (JP)

Correspondence Address: Adams & Wilks, 31st Floor, 50 Broadway, New York, NY, 10004, US

(21) Appl. No.: 10/297,809

(22) PCT Filed: Feb. 27, 2002
(86) PCT No.: PCT/JP02/01819

(30) Foreign Application Priority Data
Mar. 9, 2001 (JP) 2001-67114

(51) Int. Cl. G21G 5/00; A61N 5/00; H01J 37/302

(52) U.S. Cl. 250/492.21

(57) ABSTRACT

A signal component for scanning a processing range and a signal component for synchronizing to movement of a moving stage are superimposed on an ion beam scanning signal. Using such a scanning signal, processing is carried out while moving a sample stage. In this way, it is possible to carry out processing using a focused ion beam device in a reduced amount of time for a plurality of samples.

### TECHNICAL FIELD

The present invention relates to a focused ion beam device.

### BACKGROUND ART

Minimum processing dimensions have been getting smaller and smaller over the years, with the advancement of micro fabrication technology.

Photolithography technology used in semiconductor manufacture has been extremely successful in realizing mass production through micro fabrication and batch processing. However, with improved performance of manufactured devices, at sections where extremely detailed processing for determining performance is required it has not been possible to achieve processing of specified accuracy using the conventional photolithography technology.

Processing using photolithography technology generally has non-processed regions covered with resist, and exposed process regions are treated with a combination of sputtering using an ion beam or the like and chemical reaction using reactive gas. Since an ion beam used in such a situation is intended to realize a large quantity of processing in a short time, a beam having a wide cross section, known as a broad beam, is used.

However, with this type of method, variation in processing shape at the time of large quantity processing does not satisfy the demanded dimensional accuracy for sections that require extremely accurate processing to determine performance. For this reason, focused ion beams have been used that can correct this variation and perform more detailed processing.

A region to be processed of a sample is placed at a focused ion beam irradiation position. The sample is mounted on a sample stage, and moved to the focused ion beam irradiation position by controlling the sample stage. Micro fabrication is carried out through irradiation of the focused ion beam. After completion of fabrication, the sample stage is controlled to move the sample and place the next region to be processed at the focused ion beam irradiation position. This process is repeated.

A focused ion beam is different from a broad beam and has a small cross section. This means that for the same processing time the amount processed by a focused ion beam is less compared to a broad beam. Also, there is the problem that it is not possible to process a plurality of regions at the same time.

The following countermeasures are therefore taken to improve productivity:

(1) Focused ion beam current amount and current density are increased, and processing amount per unit time is increased.

(2) The number of times processing is carried out per unit time is increased by causing the sample stage to move at high speed.

However, with a method where the sample stage is moved to regions to be subjected to the second processing after the first processing has been completed and the second processing is carried out after the sample stage has stopped, processing can not be carried out while the sample stage is being moved. It is therefore not possible to improve productivity to that extent.

The object of the present invention is to solve these types of problems.

### DISCLOSURE OF THE INVENTION

As means for solving these problems, there is proposed a focused ion beam device for superimposing a signal component corresponding to a sample stage position on a scanning signal for a focused ion beam, and carrying out processing using the focused ion beam without stopping the sample stage, and a processing method using this focused ion beam device.

### BRIEF DESCRIPTION OF THE DRAWINGS

FIG. 1 is a schematic diagram showing the structure of a focused ion beam device of one embodiment of the present invention.

FIG. 2 is an example of a sample.

FIG. 3 is a drawing for describing a relationship between movement of the sample stage and a deflection signal.

FIG. 4 is a drawing for describing a deflection signal at a deflection electrode.

FIG. 5 is a drawing for describing a relationship between first and second devices and ion beam scanning range.

### BEST MODE FOR CARRYING OUT THE INVENTION

The present invention will be described in the following together with an embodiment.

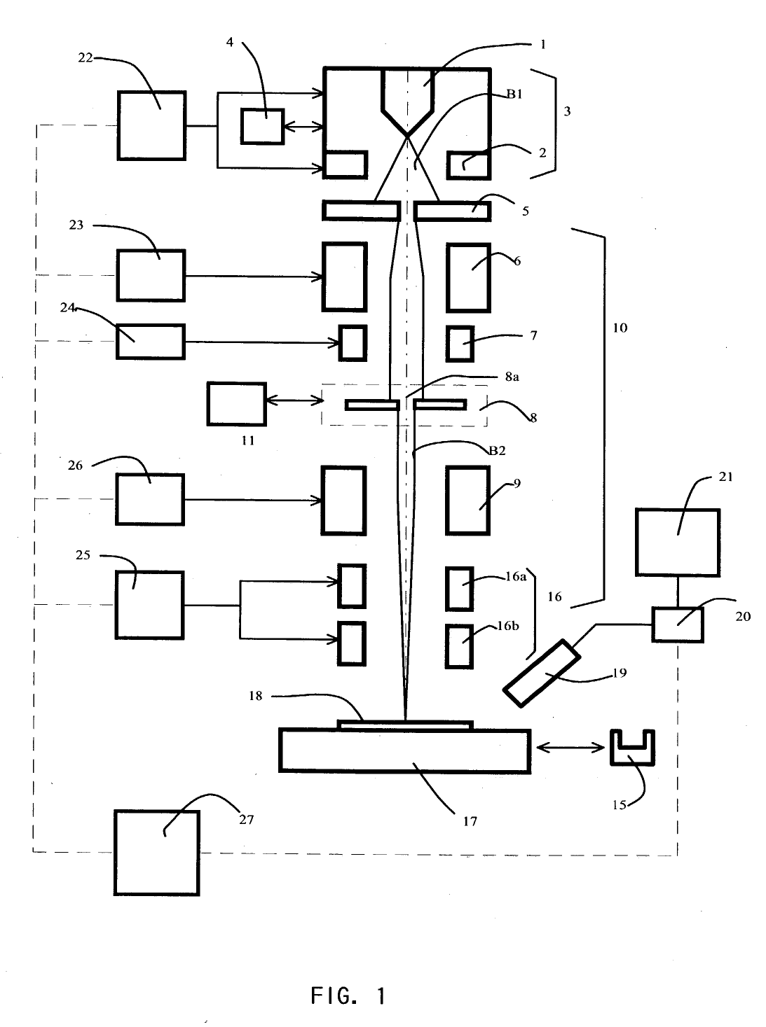

FIG. 1 is a schematic diagram showing the structure of a focused ion beam device of one embodiment. As shown in FIG. 1, an ion source section 3 having a liquid metal ion source 1, such as Ga, and an extraction electrode 2 is mounted on an XY direction movement unit 4, and is provided capable of movement in the XY directions which are the two directions orthogonal to a generated beam. A monitoring aperture 5 allowing only a central high energy density portion of a high intensity ion beam B1 generated from the ion source section 3 to pass, and measuring electric current of that captured ion beam, is located at a beam irradiation side of the ion source section 3. Also, a charged particle optical system 10 comprising a condenser lens 6, a blanking electrode 7, an aperture 8 and an objective lens 9 is arranged at an outgoing beam side of the monitoring aperture 5, and a high intensity ion beam B1 generated from the ion source section 3 is focused into a focused ion beam B2 by the charged particle optical system 9.

Here, the aperture 8 is provided with a plurality of thru-holes 8a of differing diameters that can be changed over between using the thru-hole changing device 11. That is, the aperture 8 can be changed over to one of the plurality of thru-holes 8a of differing diameter using the clear hole changing device 11. With this example, the diameter of a clear hole 8a can be changed by causing a member having a plurality of clear holes 8a of differing diameter to slide, but it is also possible to change the diameter of a single clear hole 8a continuously or in steps. The structure of the clear hole changing device 11 is not particularly limited in this respect, but the structure disclosed in Japanese Patent Laid-open No. Sho. 62-223756, for example, can be given as a specific example.

Also, the aperture 8 has radial positions of each clear hole 8a capable of being moved in the XY directions by an XY direction movement unit. With the example of FIG. 1, the clear hole changing device 11 has a function to switch clear holes 8a by sliding, which means that it can also act as the XY direction movement unit.

Blanking means made up of the blanking electrode 7 of the charged particle optical system 10 and a control power source 24 is provided, and switches the focused ion beam on and off. That is, when the focused ion beam B2 is off, the aperture 8 is closed off by applying a voltage to the blanking electrode 7 to cause the focused ion beam B2 to be deflected.

A deflection electrode 16 for scanning the focused ion beam B2 to a desired position is located at a beam exit side of the aperture 8, and the focused ion beam B2 scanned by deflection electrode 16 is irradiated to a desired position of the sample 18 mounted on the sample stage 17. The deflection electrode 16 has a two stage structure comprising a first deflection electrode 16a located at a beam outgoing side and a second deflection electrode 16b being the sample side.

A detector 19 for detecting secondary charged particles released from the surface of the sample 18 irradiated by the focused ion beam B2 is arranged above the sample stage 17. An image control section 20 for amplifying a detection signal and acquiring a planar intensity distribution of secondary charged particles and an image display unit 21 for displaying a pattern formed on the sample surface based on a planar intensity distribution signal from the image control section 20 are connected to the detector 19.

A faraday cup 15 capable of changing position with the sample stage 17 is provided beside the sample stage 17. The faraday cup 15 is irradiated with the focused ion beam B2 instead of the sample 18 in order to measure beam current.

An ion source control power source 22, condenser lens power source 23, blanking power source 24, deflection power source 25, and objective lens power source 26 for applying respectively desired voltages are respectively connected to the ion source section 3, condenser lens 6, blanking electrode 7, objective lens 9 and deflection electrode 16, and a control section 27 for synthetically controlling the entire focused ion beam device and constituted by a computer system capable of individually controlling the XY direction movement unit 4, the clear hole switching unit 11, each of the power sources 22-26 and the image control section 20 are also provided.

With this type of focused ion beam device, an ion beam B1 taken from the ion source section 3 is focused by the charged particle optical system 9, scanned by the deflection electrode 16 and irradiated to the sample 18, making it possible to process the sample 18. Although not shown in the drawings, with this example, a gas irradiation nozzle is provided close to the sample 18, and by supplying gas from the gas irradiation nozzle at the same time as irradiating the focused ion beam B2 it is possible to carry out localized film forming using beam assisted CVD.

When performing this type of processing, processing conditions can be monitored using the image display unit 21. Although again not shown in the drawings, the surface of the sample 18 is preferably illuminated by general illumination and at the same time it is possible to observe the sample surface using an optical microscope.

A sample processing method using this type of focused ion beam device will be described in the following.

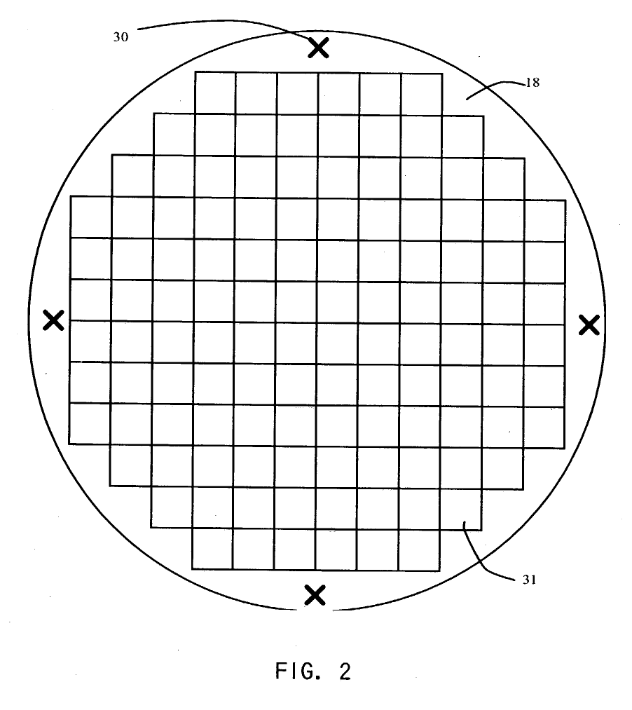

An example of such a sample is shown in FIG. 2. Devices having the same shape, such as semiconductor integrated circuits, are arranged in a grid.

A computer system 27 is input with information relating a mark 30 located on the sample 18 and position coordinates of each device 31.

The sample 18 is mounted on the sample stage 17, and a vacuum is created in a sample room not shown in FIG. 1.

A plurality of marks 30 formed at known positions in advance on the sample are sequentially moved to an irradiation range of the focused ion beam while causing the sample stage 17 to move. An ion beam scans and irradiates regions containing a mark, and a secondary charged particle image due to secondary charged particles produced at the sample surface is observed. Places to be processed of each device positioned in a grid on the sample are calculated by the computer system 27 of the focused ion beam device from coordinates for the position of the observation image of each mark 30 observed and the sample stage 17 at the time of observation

The sample stage is moved so that among the devices 31 arranged in a grid on the sample, a region to be processed of a device to be processed first enters a scanning and irradiation range of the focused ion beam.

As required, the focused ion beam is scanned and irradiated to the periphery of regions to be processed, and the regions to be processed are reconfirmed by confirming a secondary charged particle image due to secondary charged particles produced by the sample surface.

Next, blanking is canceled so that the ion beam is irradiated onto the sample, and the focused ion beam is scanned and irradiated onto the regions to be processed to carry out actual processing.

Observation and processing can be performed either with the same beam current or with differing beam currents. Generally, in order to minimize the effects on the sample, observation is carried out with a smaller beam current than when processing. Beam current is changed by setting the condenser lens 6 and switching thru-holes 8a of the aperture 8. By switching beam current, condenser lens setting voltage and position of a thru-hole 8a of the aperture 8 is adjusted in advance so that the ion beam sample irradiation position does not change. At this time, although not shown in the drawings, a deflection electrode for adjusting irradiation position and irradiation angle of the beam to the aperture clear holes 8a is provided at an ion source side of the aperture 8. When the condenser lens voltage, position coordinates of the aperture clear holes 8a and the deflection electrode are provided in this way, adjustment conditions for the setting voltage etc. are stored in the computer system 27 so that even if observation and processing are carried out with different beam currents the beam irradiation position is not changed.

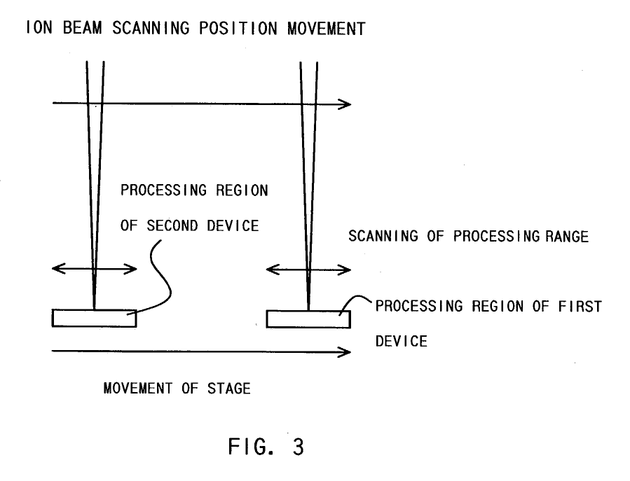

FIG. 3 is a drawing showing a manufacturing method using the focused ion beam device of the present invention. If processing is started by scanning of the ion beam to above a predetermined processing region, the sample stage 17 also starts to move. At this time, an ion beam scanning signal is made up of a signal component for scanning the processing range and a signal component for causing synchronization with movement of the sample stage. As a result, while the ion beam scans the processing region according to movement of the processing region following movement of the sample stage, the irradiation position of the ion beam is made to move in exactly the same direction and by the same amount as the sample stage.

Speed of movement of the sample stage is chosen so that processing of regions to be processed is completed before the sample moves out of the irradiation range of the ion beam.

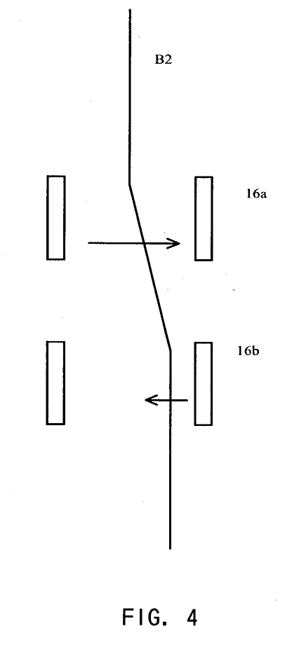

The signal component for scanning the processing range is a signal for irradiating a specified ion beam to each location on the processing region. At this time, a deflection electrode have a two stage upper and lower structure is used, and as shown in FIG. 4, by supplying a deflection signal that swings back to the lower stage deflection electrode 16b with respect to a deflection signal of the upper deflection electrode 16a, the irradiation angle of the ion beam to the sample surface does not depend on the position of the region to be processed and optimum processing is carried out.

Also, if the device shape is large it is necessary to use a peripheral section of the ion beam scanning irradiation range. Distortion of a scanned shape irradiated on the sample surface becomes large at the periphery of the scanning irradiation range compared to at a central section. In this case, a signal component compensating for distortion is superimposed on the deflection signal, making it possible to compensate for the effects of distortion.

A signal component for synchronizing to movement of the sample stage can be generated in advance by the computer system based on movement speed of the sample stage. At this time, a mark 30 on the sample is observed by ion beam scanning and irradiation at fixed intervals, and it is also possible to correct the position of the sample stage as required. Although not shown in the drawings, it is also possible to provide a sensor for detecting position of the sample stage on the sample stage and to generate the synchronization component using a sensor output signal.

In FIG. 3, once processing of a processing region of a first device is completed, a blanking signal is applied to the blanking electrode and the beam does not reach the sample.

A scanning signal for the deflection electrode is set so that the beam is irradiated on a processing region of a second device.

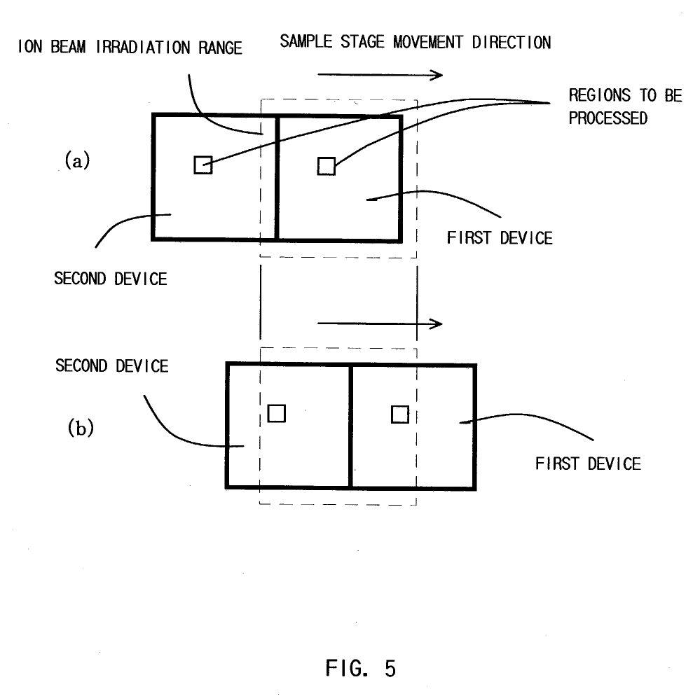

Continuing on, the blanking signal is removed, and processing for the processing region of the second device commences. FIG. 5 is a drawing for describing a relationship between the first and second devices and the ion beam irradiation range. FIG. 5(a) shows the ion beam irradiation range at a point in time when the region to be processed of the first device is being processed. In accordance with movement of the sample stage, the scanning range of the ion beam is delayed so that the region to be processed of the first device is always scanned within the irradiation range of the ion beam. FIG. 5(b) shows the situation where the sample stage and the ion beam irradiation position are moved from the positions of FIG. 5(a), processing of the first device is mostly complete, and processing of the second device commences. As shown in FIG. 5(b), before completion of processing for the region to be processed of the first device, the region to be processed of the second device is positioned in the scanning irradiation range of the ion beam to make it possible to advance processing without taking a lot of time to move the sample stage.

Also, the region to be processed for one device can actually be at a number of places.

Processing can be through sputtering etching using ion beam irradiation or through beam assisted CVD by irradiating an ion beam while spraying source material gas.

By repeating this series of processing steps, it is possible to process all regions to be processed of the devices located in a grid pattern on the sample.

### INDUSTRIAL APPLICABILITY

As described above, according to the present invention it is possible to carry out processing without being influenced by sample movement time, even in the event that a plurality of samples are processed with a focused ion beam device, which means that it is possible to improve throughput and productivity of a processing device.

It is also possible to carry out extremely high precision processing because processing position on the sample and irradiation angle of the beam to the sample are optimized.

1. A focused ion beam device for sequentially processing respective regions to be processed of a plurality of samples, comprising at least:
an ion source;
a charged particle optical system for turning an ion beam generated by the ion source into a focused ion beam;
a deflection electrode for deflecting the focused ion beam; and
a sample stage for mounting the plurality of samples, wherein
processing is carried out by scanning and irradiating the focused ion beam sequentially on the regions to be processed while causing the sample stage to move.

2. The focused ion beam device of claim 1, wherein:
a focused ion beam scanning signal has a signal component for scanning the regions to be processed, and a signal component for synchronizing with movement of the sample stage.

3. The focused ion beam device of claim 2, wherein:
the focused ion beam scanning signal has a signal component for scanning the regions to be processed, a signal component for synchronizing with movement of the sample stage, and a signal component for making an incident angle of the focused ion beam to the sample constant regardless of the position of the regions to be processed.

4. The focused ion beam of claim 3, wherein:
the signal component for making the incident angle of the focused ion beam to the sample constant regardless of the position of the regions to be processed is realized by having two deflection electrodes disposed vertically in the charged particle optical system, and causing one of the upper or lower deflection electrodes to have a scanning signal component in the reverse direction as a direction of a scanning signal applied to the remaining upper or lower electrode.

5. The focused ion beam device of claim 4, wherein:
the deflection electrodes are closer to a sample than the objective lens.

6. The focused ion beam device of claim 2, wherein the signal component for synchronizing with the sample stage is generated using an output of a sensor which is attached to the sample stage to detect a position of the sample stage.

7. A processing method, using the focused ion beam device of claim 1 to claim 6, for sequentially processing the same location of devices of the same shape disposed in a grid arrangement on a sample, comprising the steps of: positioning a region to be processed of a second device within the irradiation range of the focused ion beam before processing of the first device on the sample is completed; moving the focused ion beam to the region to be processed of the second device at the same time as completing processing of the first device to commence processing it.
